(19) United States
(12) Patent Application Publication
(10) Pub. No.: US 2006/0102935 A1
(43) Pub. Date: May 18, 2006

(54) Transistor-based biosensors having gate electrodes coated with receptor molecules

(75) Inventors: Shlomo Yitzchaik, Jerusalem (IL); Micha Spira, Jerusalem (IL); Joseph Shappir, Mevasseret Zion (IL)

Correspondence Address: BROWDY AND NEIMARK, P.L.L.C.;624 NINTH STREET, NW, SUITE 300, WASHINGTON, DC, 20001-5303, US

(73) Assignee: Yissum Research Development Company of the Hebrew University of Jerusalem, Jerusalem (IL)

(21) Appl. No.: 10/534,455

(22) PCT Filed: Nov. 11, 2003
(86) PCT No.: PCT/IL03/00941

§ 371 Date: Dec. 1, 2005

(30) Foreign Application Priority Data
Nov. 11, 2002 (IL) 152746

(51) Int. Cl. H01L 23/58

(52) U.S. Cl. 257/253; 257/414

(57) ABSTRACT

A device and method are presented for detecting analyte molecules in a medium. At least one FET (Field Effect Transistor) is provided being formed by at least one pair of source-drain electrodes and at least one gate electrode. The gate electrode is coated with a layer of receptor molecules that in the presence of said analytes catalyze a reaction that causes release of ions in a medium surrounding said receptor molecules. A monolayer of linker molecules is provided for linking said receptor molecules to said at least one gate such that a distance between the receptor molecules layer and the surface of the coated gate is smaller than 15A. In the prefered embodiments, the receptor molecules are enzymes (e.g. acetylcholine estarase) or peptides, and the analyte molecules are pesticides, herbicides and chemical pollutants of industrial origin.

### FIELD OF THE INVENTION

This invention relates to a biosensor for the detection of molecules. More specifically, the present invention relates to a biosensor that incorporates an enzyme.

### REFERENCES

Cahn, T. M. and Jackson, S. A., Biosensors, Chapman & Hall, Paris, 1991.
Coulet, P. R., “From chemical sensors to bioelectronics: a constant search for improved selectivity, sensitivity, and miniaturization”, Proceedings of the scientific Computing and Automation (Europe).
Gopel, W., Heiduschka, P., Introduction to bioelectronics: interfacing biology with electronics, Biosens., Bioelectron., 1994, iii-xiii.
Gorchkov, D. V., Soldatkin, A. P., Poyard, S., Jaffrezic-Renault, Martelet, N.,C., Application of charged polymeric materials as additional permselective membranes for improvement of the performance characteristics of urea-sensitive enzymatic field effect transistors: 1. Determination of urea in model solutions, Mater. Sci. Eng., C 5, 1997, 23-28.
Jimenez, C., Bartoli, J., de Rooij, N. F., Koudelka-Hep, M., Use of photopolymerizable membranes based on polyacrylamide hydrogels for enzymatic microsensor construction, Anal. Chim. Acta. 351, 1997, 169-176.
Kharitonov, A. B., Shipway, A. N., Willner, I., An Au-nanoparticle bis-bipyridinium cyclophane-functionalized ion-sensitive field-effect transistor for the sensing of adrenaline, Anal. Chem. 71, 1999, 5441-5443.
Kharitonov, A. B., Zayats, M., Lichtenstein, A., Katz, E., Willner, I., Enzyme monolayer-functionalized field-effect transistors for biosensor applications, Sensors and Acuators B., 70, 2000, 222-231.
Powner E. T., Yalcinkaya F., Intelligent Biosensor, Sensor Review, Vol. 17, No.2,1997, pp. 107-116.
Senillou, A., Jafferezic-Renault, N., Martelet, C., Cosnier, S, A miniaturized urea sensor based on the integration of both ammonium based urea enzyme field effect transistor and a reference field effect transistor in a single chip, Talanta 50, 1999, 219-226.
Sze, S. M., Semiconductors Sensors, Wiley, New York, N.Y., 1994, pp. 127-8.
Turner, A. P. F., Karube, I. And Wilson, G. S., Biosensors, Fundamentals And Applications, Oxford University Press, Oxford, 1987, p. 5.
Wise, D. L., Applied Biosensors, Butterworths, London, 1989, pp. 93-114.

### BACKGROUND OF THE MENTION

The Integration of biologically active molecules with electronic transducers has emerged as an elegant and effective way of creating high fidelity systems for the detection of a wide range of biological activities (Turner, 1987; Gopel, 1994; Cahn, 1991). The aim of such biological sensory systems is the production of an electrical signal which is proportional to the concentration of a certain biochemical agent, and thus reflects the level of biochemical activity of the biocatalyst involved (Powner, 1997). Such systems serve as translators of biological events into electrical signals and can prove to be the link between the much-understood world of silicon-based electronics and the biological world.

The high specificity of biomolecules such as enzymes, antibodies, etc. allows for the creation of reaction-specific biosensory systems that can be used for a wide array of applications (Coulet). A review of sensor technology may be found in Sze (1994). One type of sensor technology prepared in the past concerns the use of ion-sensitive field-effect transistor (ISFET) in which the normal metal-oxide-silicon field-effect transistor (MOSFET) gate electrode is replaced by an ion-sensitive membrane with the ability to detect ion concentrations in solution (Wise, 1989), as schematically shown in FIG. 1.

Enzyme-based sensory systems such as the traditional enzyme-based field-effect transistors (ENFET) and enzyme-electrodes have also been described in the past (Jimenez, 1997; Senillou, 1999; Gorchkov, 1997; Kharitonov, 1999, 2000).

### SUMMARY OF THE INVENTION

The inventors have found that a Field Effect Transistor (FET) may be used as a sensor for molecules in solution and air, and may be used specifically to monitor catalytic activity of an enzyme assembled thereon. This is achieved by coating a gate electrode of the FET with a layer of receptor molecules that in the presence of certain analytes can catalyze a reaction that causes release of ions in a medium surrounding said receptor molecules, and providing a monolayer of linker molecules for linking said receptor molecules to said gate such that the distance between the receptor molecules layer and the surface is smaller than 15 Å. Preferably, this distance is of about a few angstroms.

Thus, according to one aspect of the present invention, there is provided a device for the detection of analyte molecules, the device comprising at least one pair of source-drain electrodes and at least one gate electrode to thereby define at least one Field Effect Transistor (FET), wherein said at least one gate electrode is coated with a layer of receptor molecules that in the presence of said analytes catalyze a reaction that causes release of ions in a medium surrounding said receptor molecules, and a monolayer of linker molecules is provided for linking said receptor molecules to said at least one gate such that a distance between the receptor molecules layer and the surface of the coated gate is smaller than 15 Å.

The receptor molecules are preferably enzymes or peptides, and more preferably enzyme molecules. One specifically preferred enzyme is acetylcholine esterase (AChE).

Thus, according to another broad aspect of the invention, there is provided device for the detection of analyte molecules, the device comprising at least one air of source-drain electrodes and at least one gate electrode to thereby define at least one Field Effect Transistor (FET), wherein said at least one gate electrode is coated with a layer of receptor molecules including acetylcholine esterase (ACHE) that in the presence of analytes including acetylcholine catalyzes a reaction that causes release of ions in a medium surrounding said receptor molecules, and a monolayer of linker molecules is provided for linking said receptor molecules to said at least one gate such that a distance between the receptor molecules layer and the surface of the coated gate is smaller than 15 Å, said linker molecules being selected from conjugated or unconjugated aliphatic, aromatic or heteroaromatic molecules, having at least one functional group capable of covalently binding to said surface and at least one functional group capable of covalently binding to said receptor molecules.

The analyte molecules to be detected by the device of the present invention may be those selected from chemical agents used in agriculture, in environmental applications, industry and chemical warfare. The chemical agents are pesticides, herbicides, nerve agents and synthetic or natural toxins emitted from industrial plants.

The Field Effect Transistor is an Ion Sensitive Field Effect Transistor. The gate electrode is an ion sensitive oxide gate. The ion-sensitive oxide is preferably Aluminum Oxide (Al₂O₃), Silicon Nitride (Si₃N₄), Indium Tin Oxide (In₂O₃—Sn₂O₃), Silicon Oxide (SiO₂) or Tantalum Oxide (Ta₂O₅).

The device may include an array of differently coated gate electrodes, which may be associated with the same source-drain pair, or with different source-drain pairs.

According to another aspect of the present invention, there is provided a method of detecting analyte molecules in a medium, the method comprising:
(a) providing at least one Field Effect Transistor (FET) formed by a source-drain electrode pair and at least one gate electrode that is coated with a layer of receptor molecules that in the presence of certain analytes catalyze a reaction that causes release of ions in a medium surrounding said receptor molecules, and a monolayer of linker molecules for linking said receptor molecules to said at least one gate such that a distance between the receptor molecules layer and the surface of the coated gate is smaller than 15 Å.
(b) accommodating said at least one FET such that said at least one gate is exposed to a medium suspected of containing analyte molecules capable of reacting with the receptor molecules, thereby affecting a release of ions in said medium, and
(c) monitoring a change in an electric current between the source and drain electrodes caused by the release of ions, said change being indicative of the presence of said analyte in the medium, thereby enabling measuring the analyte concentration in the medium.

The medium may be one of the following: water, sea water, buffer, and ionic solution.

Abbreviations

ISFET—Ion-sensitive field effect transistor; CyC—Cyanuric chloride; I_{ds}—drain-source current; TSA—topotactic self-assembly; ACh—Acetylcholine; AChE—Acetylcholine esterase; DTNB—5,5′-dithio-bis (2-nitrobenzoic acid); TNB—thionitrobenzoic acid.

### BRIEF DESCRIPTION OF THE DRAWINGS

The present invention will be more fully understood and appreciated from the following detailed description, taken in conjugation with the examples and drawings, in which:

FIG. 1 shows a standard metal-oxide-silicon field-effect transistor (MOSFET, left) and an Ion-Sensitive Field Effect Transistors (ISFETs, right). In an ISFET the metal oxide gate electrode is replaced by an ion sensitive membrane by which changes in ionic concentrations in solutions induce a change in ISFET transduction that can be measured by drain-source current (I_{ds}).

FIG. 2 shows the stepwise construction of the layered structure. In step (A), Cyanuric Chloride is reacted with the oxide layer of the ISFET gate surface to form covalent linkage. In step (B), the covalently bound cyanuric chloride layer forms a covalent linkage (arbitrary Lysine residue of enzyme) the enzyme.

FIG. 3 shows the experimental (ellipsometry data taken at 75°) ψ and Δ as a function of wavelength for a monolayer of CyC on Silicon substrate. Based on the complete fitting between the model and experimental results as shown in this Figure, the thickness of the layer was determined.

FIG. 4 represents the hydrolysis of acetylcholine to choline in the presence of AChE in water. The reaction results in the generation of acetic acid and protonation of the solution.

FIG. 5 shows the Acetylcholine dose response of the assembeled ACHE-FET structure.

FIG. 6 shows the ACHE inhibition by eserine as detected by the AChE-FET structure of the present invention.

FIG. 7 shows the response of the structure of the present invention to application of carbamylcholine in comparison with Ach.

FIG. 8 exhibits the ability of the ACHE-FET structure to detect small Ach quantities through Ach iontophorsis.

FIG. 9 shows the UV-vis absorption spectrum of TSA derived CyC monolayer on quartz substrate.

FIG. 10 shows the absorption spectra of 1.25×10⁻⁴ M of ACh and 5×10⁻⁵ M DTNB solution before (having a peak at 325 nm) and after (having a peak at 410 nm) a 15-minute exposure to ACHE containing substrate.

FIG. 11 shows the absorption spectra of 1.25×10⁻⁴ M ACh and 5×10⁻⁵ M DTNB solution exposed to AChE containing substrate, recorded in-situ at 20-second interval.

FIG. 12 represents the optical density at 410 nm versus time following the insertion of a glass substrate containing immobilized ACHE into 2.5×10⁻⁴M ACh and 5×10⁻⁵ M DTNB solution.

FIG. 13 shows Reaction velocity verses ACh concentration for the surface-bound ACHE.

FIG. 14 represents a graphical determination of Kₘ and Vₘₐₓ for the surface bound AChE.

FIG. 15A-B show substrate dependency of ISFET responsiveness: (A) Ach-Iodine dosage response, and (B) basic characterization shows a substantial change in the ISFET transduction and amplification (dI/dV).

FIGS. 16A-B show the effects of AChE inhibition by Eserine on I_{ds}. (A) Eserine was injected to the buffer solution at t=75 and at t=400 sec. Acetylcholine was not washed from solution. A major decrease in I_{ds} of the enzyme modified ISFET was observed and a return to a lower I_{ds} level occurred within 50 sec of application. (B) Eserine had very little effect on non enzyme modified ISFET. Acetylcholine was injected at t=230 and at t=400 sec and showed I_{ds} increase. Eserine was injected at t=610 sec and showed similar effects, suggesting response of non-enzyme modified ISFET is merely an artifact.

FIG. 17 depicts the ISFET long term fidelity. AChI-induced response has been measured after 1 month at 4° C. and no apparent deterioration of enzyme layer was observed. (AChI was applied at t=50 and at t=160 sec.).

### DETAILED DESCRIPTION OF THE INVENTION

In the following, the invention will be illustrated in reference to some non-limiting specific embodiments.

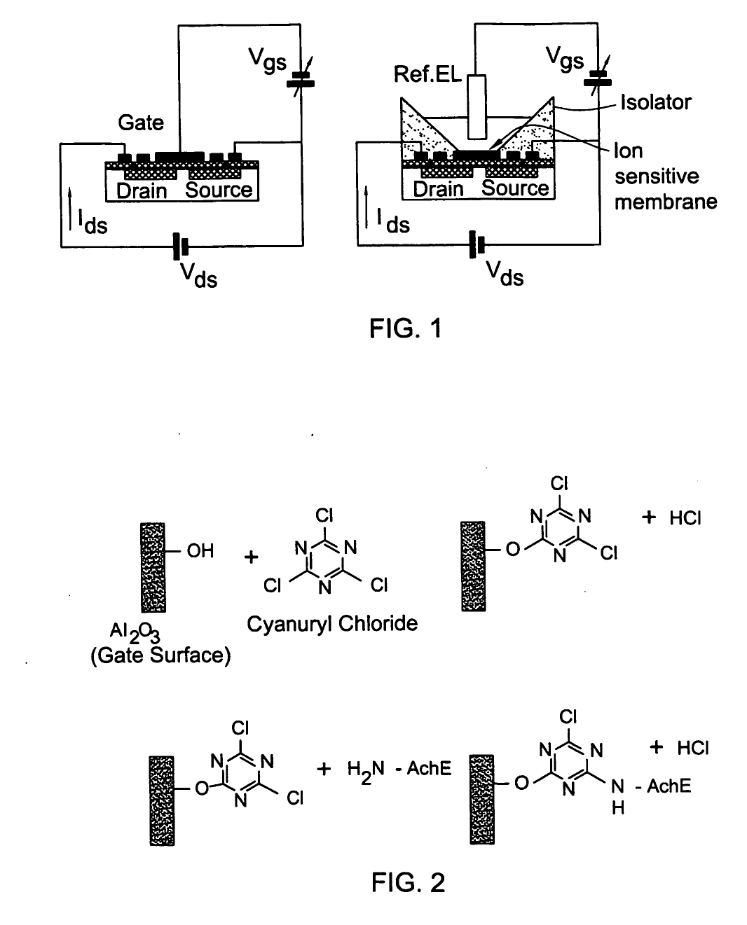

FIG. 1 illustrates MOSFET and ISFET structures suitable to be used in a device of the present invention for the detection of molecules. Generally, the device of the present invention is a FET, in which a gate electrode is formed with a layer of enzyme molecules capable of catalyzing a reaction that causes release of ions in a media surrounding said enzyme, and a monolayer of linker molecules linking said enzyme to said gate such that the distance between the enzyme and the surface is smaller than 15 Å.

The media surrounding said enzyme may be air, water, sea water, buffer solution, ionic solution and others.

FIG. 2 exemplifies how a conventional FET (ISFET in the present not limiting example) can be modified to obtain the device of the present invention. As shown, the gate surface layer Al₂O₃ is coated with cyanuric chloride (constituting linking molecules) which covalently binds to the oxide atoms and is then reacted with the ACHE (constituting receptor molecules) which binds covalently to the linking molecules through one of the reactive functional groups of the enzyme.

The enzyme may be a natural or synthetic, preferably selected from the following: proteases, lipases, RNases, DNases, peptidases, glucose oxidase, urease, chymotrypsin, butyrylcholine esterase and acetylcholine esterase. More preferably, the enzyme is acetylcholine esterase, herein designated AChE.

The ion sensitive oxide coat (gate surface layer) may be Aluminum Oxide (Al₂O₃), Silicon Nitride (Si₃N₄), Indium Oxide-Titanium Oxide (In₂O₃—TiO₃), Silicon Oxide (SiO₂) or Tantalum Oxide (Ta₂O₅).

The linker molecules are positioned between and covalently bound to both the oxide-coat of the gate and the receptor molecules layer (e.g., enzyme). The linker molecules are preferably selected from conjugated or unconjugated aliphatic, aromatic or heteroaromatic molecules, having at least one functional group capable of covalently binding to said surface and at least one functional group capable of covalently binding to said receptor molecules (enzyme). The term “heteroaromatic” refers to aromatic compounds containing one to three heteroatoms selected from N, O and/or S. The heteroaromatic molecules are for example, and without limiting to pyridyl, pyrrolyl, furyl, thienyl, imidazolyl, oxazolyl, quinolinyl, thiazolyl, pyrazolyl, 1,3,4-triazinyl, 1,2,3-triazinyl, benzofuryl, isobenzofuryl, indolyl, imidazo[1,2-a]pyridyl, benzimidazolyl, benzthiazolyl, benzoxazolyl, and quinazolinyl.

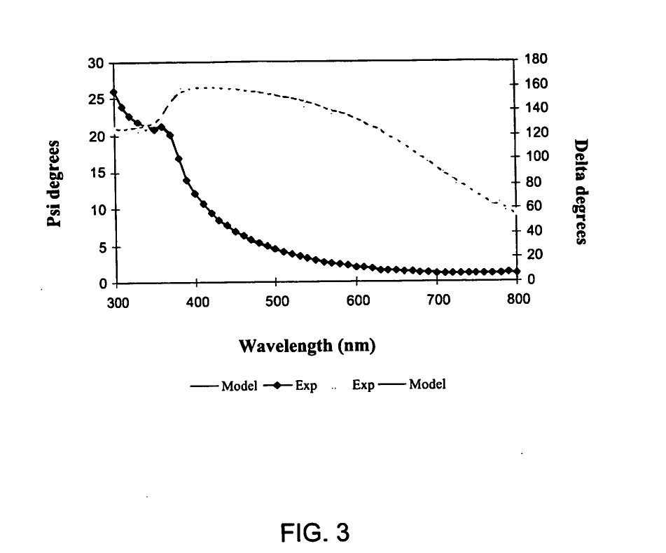

These linker molecules bind the receptor molecules to the surface such that the distance between the receptor and the surface is less than 15 Å, preferably less than 10 Å and most preferably less than 5 Å. The thickness of the layer may be determined by various methods, one of which being as shown in FIG. 3.

Such linker molecules are for example and without being limiting to short chain aliphatic molecules or mono- or polycyclic aromatic or heteroaromatic molecules capable of forming a single compact layer on the surface of the gate. Surface binding functional groups may for example be halides, activated halides, trichlorosilanes, trialkoxysilanes or other similar groups capable of binding covalently to the surface of the device.

Receptor molecules' bonding groups, capable of binding to functional groups of the receptor molecules may for example be, without being limiting to, halides (i.e. I, Br, Cl), aldehydes, carboxylic acids, active esters, acyl halides and ketones.

The linker molecules are preferably heteroaryl compounds substituted by at least one surface-binding functional group and additionally by at least one enzyme-binding functional group; both functional binding groups are preferably labile groups.

The FET device according to the invention serves as an amplifier that translates the presence and concentration of the analyte molecules (i.e. molecules being tested for) on its surface into a change in the electrical current between the source and drain, I_{ds}.

The device of the present invention utilizing an ISFET operates in the following manner. When the receptor molecules (enzyme) on the top of the ion-sensitive layer of the ISFET is brought in contact with an inhibitor of the receptor molecules, free hydrogen ions are formed (as a reaction result), the surface potential on ion sensitive layer changes, thus influencing the current I_{ds} between the drain and source, which makes this current I_{ds} directly related to the pH. The current changes can be detectable either by using a reference electrode screened from the environment (e.g., from the analyte molecules), or by utilizing a threshold-based programming means.

Enzyme-catalyzed reactions may alter the pH at the ISFET gate surface, either positively, by the uptake of protons, or negatively, by the generation of protons. Such pH changes result in an electrical activity at the gate surface of the transistor and induce current changes between the drain and source electrodes, when the gate-source potential is kept constant.

In one embodiment of the present invention, the ISFET device comprises a an aluminum oxide (Al₂O₃) gate which is covered with a layer of cyanuric chloride molecules and a layer of the ACHE enzyme being covalently bound to the cyanuric chloride layer.

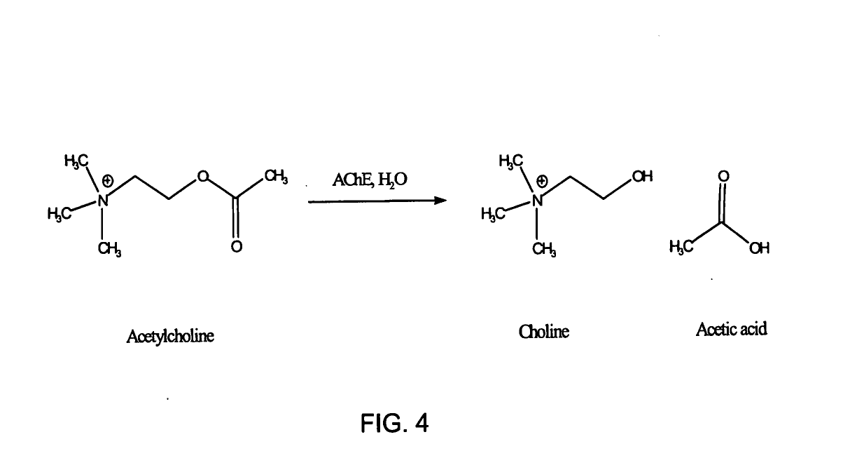

ACHE catalyses the hydrolysis of acetylcholine, resulting in the generation of acetic acid and choline as shown in FIG. 4. The generation of acetic acid and the acidification of the buffer solution induce a pH change that is recorded by the ISFET.

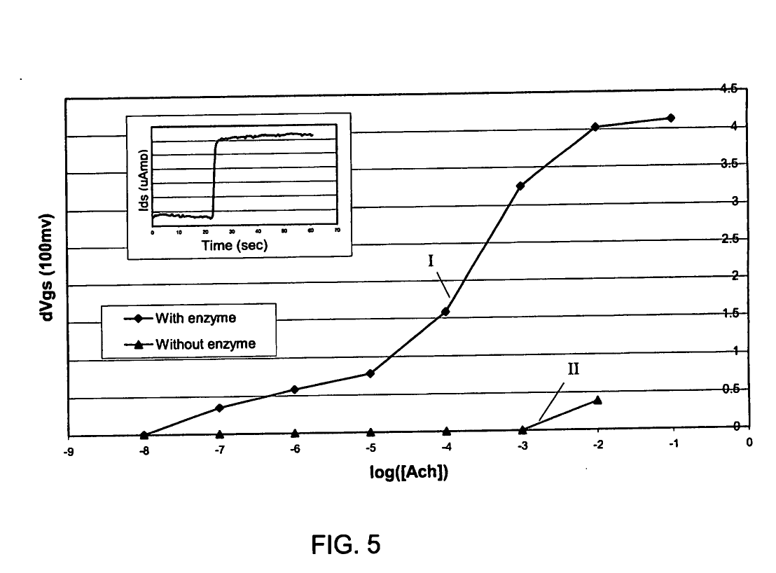

FIG. 5 illustrates experimental results of the Acetylcholine dose response of the ACHE-FET structure of the present invention. In this experiment, the ISFET has undergone covalent bonding with acetylcholine-esterase using the aforementioned cyanuryl chloride techniques. As shown in the figure, the response of the ACHE-ISFET to various doses of acetylcholine (graph I). Acetylcholine was manually applied into a solution of 5 ml Phosphate Saline Buffer (PBS) in which the ISFET resided. The response is sigmoid-shaped and clearly correlates with normal enzyme kinetics: the lowest concentration detectable thus far is 10⁻⁸ M, while saturation of response has been reached at approximately 0.05M. Bare ISFETs, that haven't undergone the ACHE bonding process showed little if any response to the application of Acetylcholine (graph II), excluding response to high concentrations (>0.1M), which might be a consequence of changes in ionic strength of the entire solution or spontaneous Ach hydrolysis. (fast response time of about 2 sec shown in the insert, dose is 0.001 ACh).

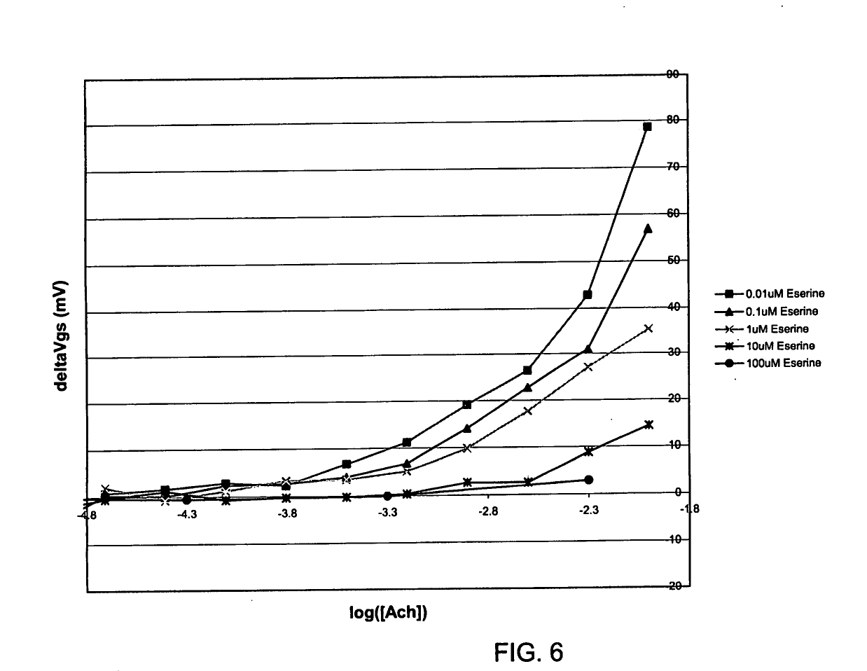

FIG. 6 illustrates experimental results of Acetylcholine esterase Inhibition by Eserine detected by ACHE-ISFET. Increasing doses of acetylcholine were applied in varying concentrations of Eserine (a reversible and competitive ACHE inhibitor) in solution. Total inhibition is observed in 100 μM Eserine. The same ACh dose response analysis in lower concentrations of Eserine shows a distinct recovery of ISFET voltage in response to Ach. Response in the presence of 0.01 μM Eserine resembles response without inhibitor (not shown). Thus, the structure of the present invention is able to detect levels of AChE Eserine inhibition in the range of 0.0 μM to 100 μM.

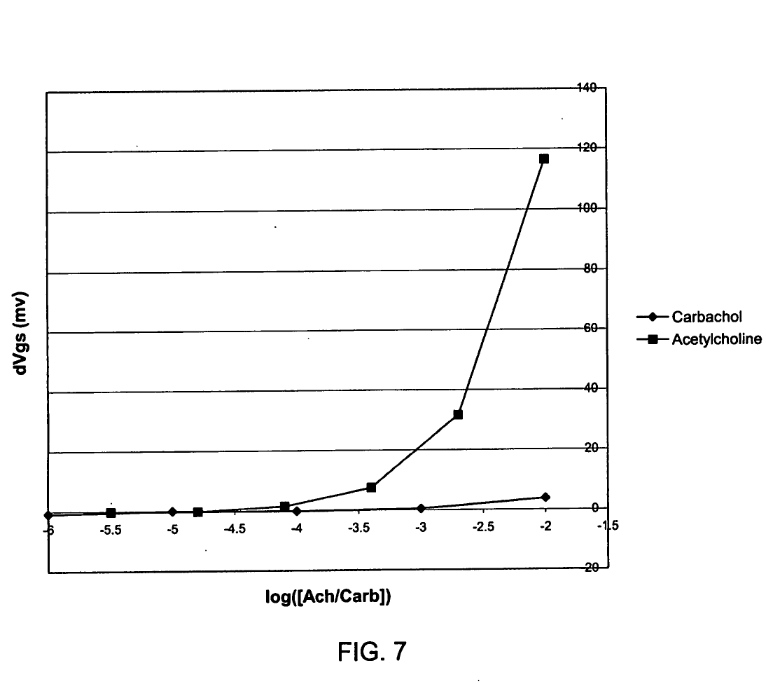

FIG. 7 illustrates the results of exposing the structure of the present invention to Carbamylcholine which produces no response in comparison to application of ACh. Carbamylcholine (C₆H₅ClN₂O₂) acts as a cholinergic agonist that is resistant to the action of cholinesterases. When applied to the solution in which the ACHE-ISFET resides, no response is evoked, in contrast to the full-scale response evoked by the application of acetylcholine. This is indicative of that the sensor of the present invention is capable of specifically detecting ACh in solution. Both carbamylcholine and acetylcholine have been dissolved in phosphate buffered saline (PBS).

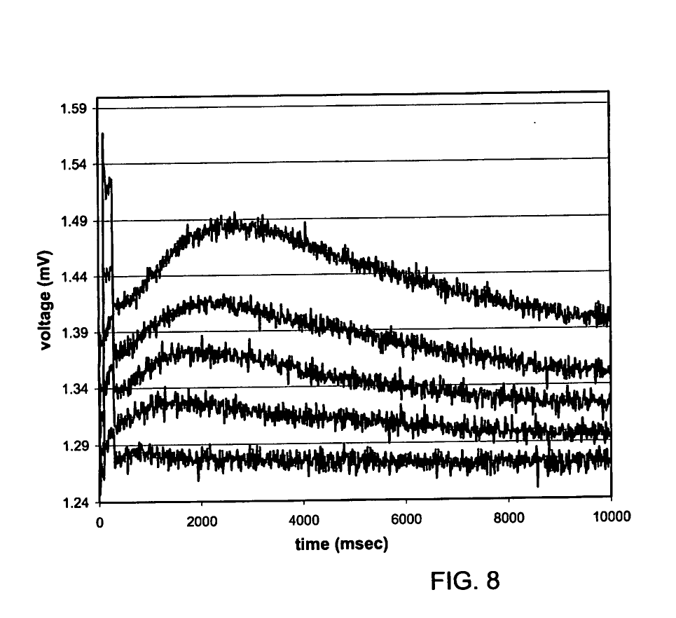

FIG. 8 illustrates the results of ACh Iontophoresis onto AChE-ISFETs. Iontophoresis experiments were conducted using the ISFETs of the present invention that have undergone the ACHE bonding process. ACh was inserted into a glass micropipette, which was then brought within distance of approximately 5 um from the gate surface of the ISFET. Negative current pulses (200 msecs) were then applied onto the micropipette with increasing amplitudes, thus releasing doses of ACh in increasing size. A constant (DC) positive current was applied to prevent leakage of Ach from pipette, and prevent depolarization. In contrast to negative pulses that resulted in the release of Ach and the ISFET's response, negative pulses did not result in a similar or reverted response (not shown). This shows that the ISFET responds specifically to Ach, and furthermore, demonstrates its ability to detect small and local Ach release. Iontophoresis experiments using bare ISFETs resulted in little or no response. Using standard Ohm's-Law calculations, ISFET's peak sensitivity has been determined to be approximately 20,000 Ach molecules. This is indicative of the capability of the structure of the present invention for sensing quanta release beyond a certain threshold.

Examples of the various applications of the device of the present invention, without being limited to are: (1) detection of pesticides and herbicides in agriculture, (2) detection of residual natural and/or synthetic toxins, pesticides and/or herbicides in water, (3) detection of residual natural and/or synthetic toxins, pesticides and/or herbicides in food and food products, (4) detection of synthetic toxins emitted from industrial plants in the air and water, (5) detection of chemical warfare agents, and (6) detection of AChE inhibitors, or agonists.

By utilizing an array of differently coated gates, either associated with the same source-drain pair, or relating to different FETs, such that each gate is composed of a different receptor molecules layer, different analyte molecules can be detected. The invention further relates to a method of detecting analyte molecules and measuring their concentration in air or in solution, e.g., water, sea water, buffer or ionic solution. The device or an array thereof is exposed as disclosed hereinbefore to a medium suspected of containing analyte molecules capable of reacting with the receptor molecules. The change in the current measured at a constant or variable electric potential applied between the source and drain is monitored, and the presence of said analyte is determined.

The determination may be qualitative, although the extent of change may serve as a quantitative measure for the level of said analyte in the medium.

The invention will be further illustrated by the following non-limiting examples.

### EXAMPLES

General

Acetylcholine esterase (C1682, taken from electric eel), acetylcholine-iodine, acetylcholine-chloride, cyanuric chloride and eserine (physostigmine), ere purchased from Sigma and were used as supplied.

Measurements were taken in a standard phosphate buffer (PBS), and hysiological solutions at room temperature.

### Example 1

### Solid-State Assembly of Cyanuric Chloride (CyC) on Glass, Quartz and Silicon

In order to study the structure-activity relationship and enzyme activity, chemisorption of cyanuric chloride (CyC) was carried out on glass, quartz and silicon wafers. The chlorides of the CyC are very labile and can undergo fast nucleophilic substitution reaction with the substrate (hydroxy containing surfaces) via topotactic self-assembly (TSA).

In this method the substrate (1 inch², active area of 1 mm²) is positioned on a spin-coater holder and wetted with a 0.1 M solution of CyC dissolved in dichloromethane. Spinning at 4000 rpm for 30 seconds resulted in a physisorbed layer of about 60-80 nm in thickness. The covalent bonding onto the surface is achieved by introducing the coated substrate into a vacuum oven (3 mTorr) at 74° C. for 10 minutes. These conditions are the optimized balance between the surface reaction kinetics and the sublimation rate of CyC. As the TSA assembly is a self-cleaning solvent-less surface reaction, a mono-molecular layer is obtained.

Contact angle measurement of water on the substrate after monolayer assembly gave a wetting angle of −77° as compared with the low wetting angle of −15° obtained with the clean unassembled substrate. Such a high contact angle is characteristic of a hydrophobic interface lacking the ability to form hydrogen bonds with the water droplet. Additionally, it shows a pinhole free monolayer coverage with no hydrophilic interaction with the under laying substrate.

Variable angle spectroscopy ellipsometry (VASE) of the CyC monolayer showed a thickness of 6.7 Å for this layer on top of 18 Å thick oxide layer. The derived ellipsometric thickness suggests that the alignment of the coupling molecule is perpendicular to the surface and is composed of a single monolayer.

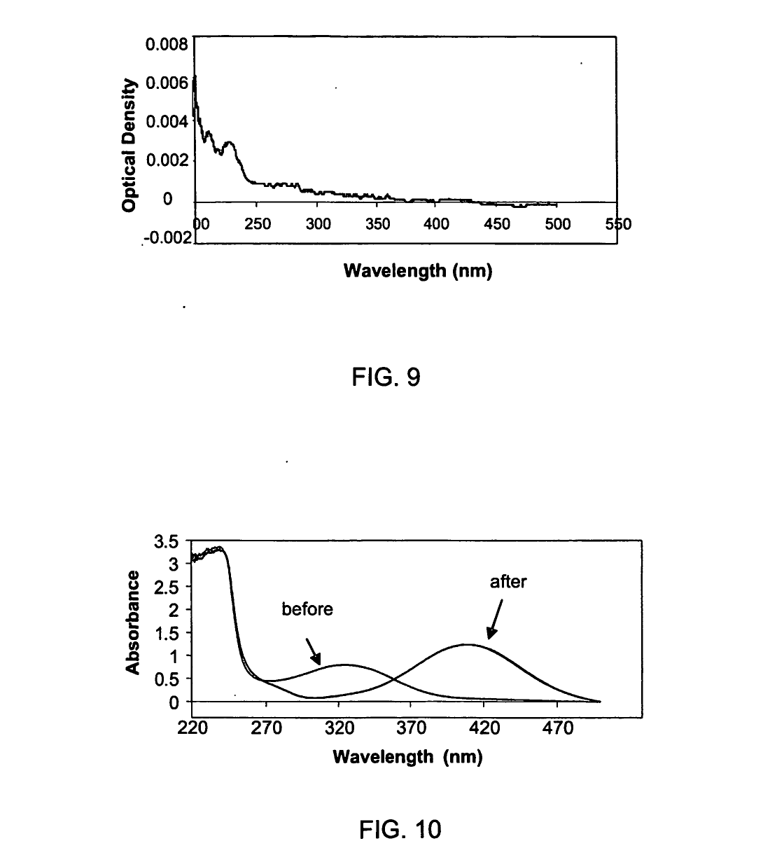

FIG. 9 shows the UV-Vis absorption spectrum of the CyC monolayer. The λₘₐₓ at 230 nm corresponds to the CyC absorption while the OD suggests a molecular number density in the order of 10¹⁴ molecules/cm².

### Example 2

### Surface Anchoring of Acetylcholinesterase (AChE)

A stock solution of the enzyme is prepared by mixing 10 μl ACHE with 100 μl PBS buffer at pH=7.4. The condensation reaction with the enzyme is obtained by placing a 10 μl the enzyme solution on the CyC containing substrate in a covered Pettri dish for 10 h at 16° C. The unreacted enzyme is then washed off with PBS buffer at pH=7.4, three times. Substrates containing immobilized enzyme were kept under buffered solution at 16° C. prior performing the various characterization tests.

To examine whether the immobilization procedures rendered the enzymatic activity, the following experiments were performed.

(a) Enzyme Activity by Ellman's Method.

In this test, the enzyme activity is measured by following the increase of yellow color produced from the reaction of thiocholine with the DTNB ion (Sawada, O., Ishida, T., Kihachiro, H., J. Biochem., 129, 2001, 899-907).

The reaction of acetylthiocholine-iodide with DTNB (5,5′-dithio-bis (2-nitrobenzoic acid) marker was conducted by immersing the solid substrate containing the immobilized ACHE, obtained above, in an optical cell compatible with the spectrophotometer. The rate of color production was measure at 410 nm. All of the investigated solutions were freshly prepared: ACh in phosphate buffer of pH=8.0 and the DTNB dissolved in PBS buffer of pH=6.5.

The enzyme activity was probed by two methods: Ex-situ and In-situ experiments. In the Ex-situ experiments the solution of the marker containing ACh is measured before and after the exposure to the immobilized ACHE substrate. The optical cell contains a solution composed of 2 ml of 2.5.10⁻³ M ACh and 2 ml of 1.10⁻⁴ M DTNB. The optical spectra were recorded before and after 15 min of the immobilized enzyme solution insertion to this optical cell. In the In-situ experiments, the substrate is immersed in the optical cell containing various concentrations of the DTNB/ACh solution. The hydrolysis product was followed in two ways: (a) by recording the spectra in fixed time interval and (b) by the time course mode at 410 nm.

FIG. 10 shows the appearance of the 410 nm peak of TNB, the reaction product of DTNB and thiocholine and the absorption of DTNB at 325 nm before the exposure to the enzyme containing substrate.

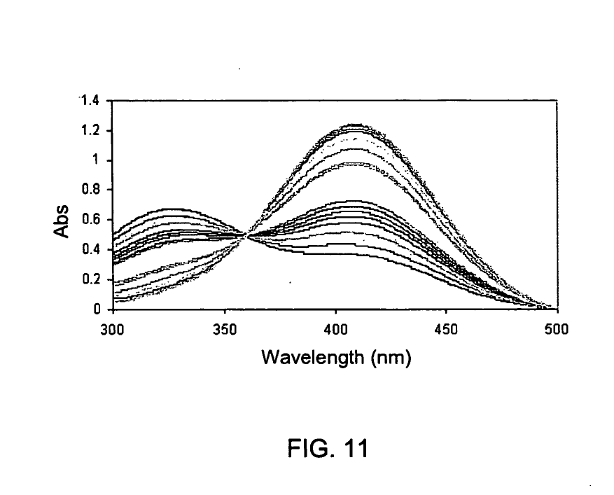

FIG. 11 demonstrates the progress of the reaction by the decrease in DTNB absorption and the increase in TNB absorption. The existence of an isosbestic point at 360 nm confirms the direct transformation between the two species.

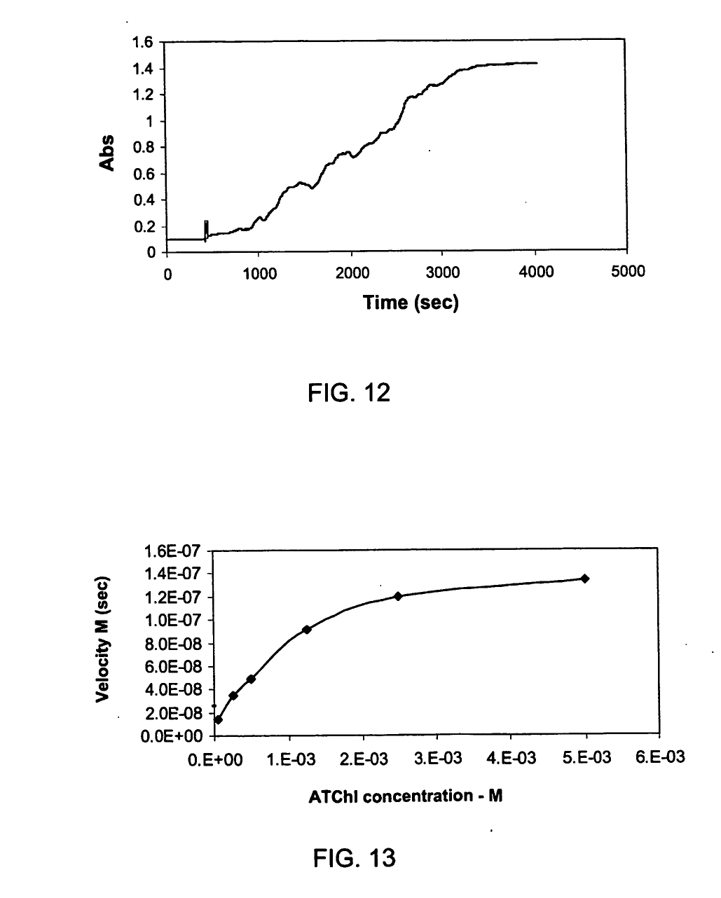

The determination of ACHE activity was conducted by probing in real time the absorption of the TNB product at 410 nm. FIG. 12 exemplified the bio-catalytic activity of the surface bound ACHE on the hydrolysis of ACh (in a given concentration) to yield thiocholine. The hydrolysis kinetics is characterized by an initial fast hydrolysis (“the linear regime”) that levels-off with the total consumption of the marker by the hydrolysis product. The slope of the “linear” part (ΔA/Δt) can yield the reaction velocity in M/sec.: where ΔA=ε×ΔC×1 (ε=14150 M⁻¹.cm⁻¹ at 412 nm and =1 cm).

(b) Reaction Velocity

Repeating the experiment with different ACh concentrations was conducted in order to give the reaction velocity dependence on the substrate concentration as shown in FIG. 13. In these experiments the DTNB concentration was kept constant (5×10⁻⁵ M).

This bio-catalytic activity of the surface-bound ACHE fits the Michaelis-Menten model for enzyme kinetics. At constant enzyme concentration the reaction velocity reaches a saturation value, which is defined as Vₘₐₓ. This is consistent with the fact that the number of active sites in the sample is constant and can't react faster with the increase in substrate concentration.

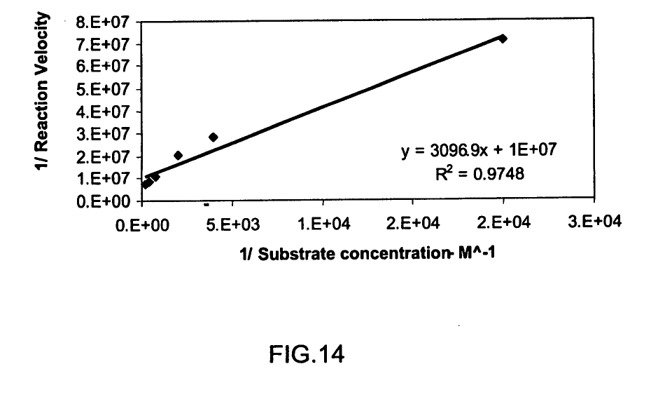

Michaelis-Menten model's defines Kₘ as the substrate's concentration that yields half the velocity of Vₘₐₓ. A Lineweaver-Burk plot, shown in FIG. 14, was used for the graphical extraction of these kinetic parameters (see Figure G). The linear regression of the data in the Lineweaver-Burk plot yield: Kₘ=3.1×10⁻⁴M and Vₘₐₓ=1×10⁻⁷M sec⁻¹, it is worth noting that these values are highly dependent on the experimental conditions such as pH, temperature and ionic-strength.

These tests indicate that the covalent assembly of AChE to a glass substrate via CyC coupling layer preserved the bio-catalytic activity of the enzyme towards the hydrolysis of ACh. This may be concluded from the Vₘₐₓ and Kₘ values that are comparable to those of the free enzyme in solution.

### Example 3

### ISFET Device Fabrication

ISFETs were first rinsed with isopropanol and dried under Argon. A is solution of 0.1M cyanuric chloride (in dichloromethane) was prepared and then applied to the Al₂O₃ gate surface of the ISFET. ISFETs were then dried with Argon and heated at 70° C. for 15 minutes, then rinsed again with dichloromethane and dried under Argon. Acetylcholine esterase, 0.1M, was applied onto the modified gate surface and left for 1 hr at room temperature, and then rinsed with PBS.

### Example 4

### The Measurements

The resulting hybrid system of Example 3, was immersed in a PBS solution as a background electrolyte for the measurements. A standard Ag/AgCl electrode was used as the reference electrode. The current between source and drain electrodes (I_{ds}) was measured, while potential between drain and source electrodes (V_{ds}), and between the gate and source electrodes (V_{gs}) were kept constant at 0.1V and 0.45V, respectively, recording the electrical activity occurring at the gate surface.

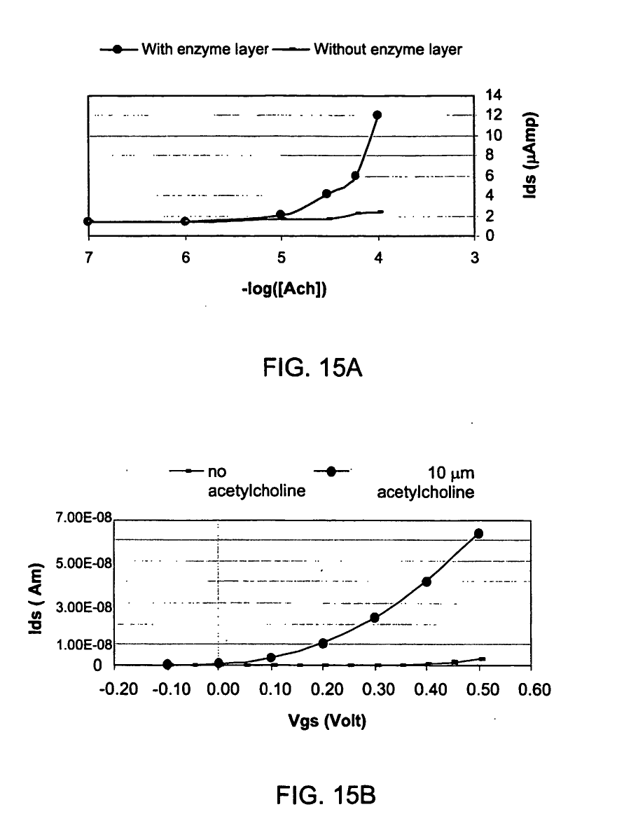

The substrate dependence of the drain-source current recorded by the modified ISFET correlates with normal enzyme activity analysis and is shown in FIG. 15A. The modified ISFET showed responsiveness at concentrations of as low as at 10⁻⁸ M. The response plateau is observed at concentrations in the range of 1 mM (not shown). Basic characterization of ISFET transduction was carried out before and after ACh-Iodine or ACh-chloride application (FIG. 15B). A substantial increase in ISFET transduction was observed in correlation with dosage response.

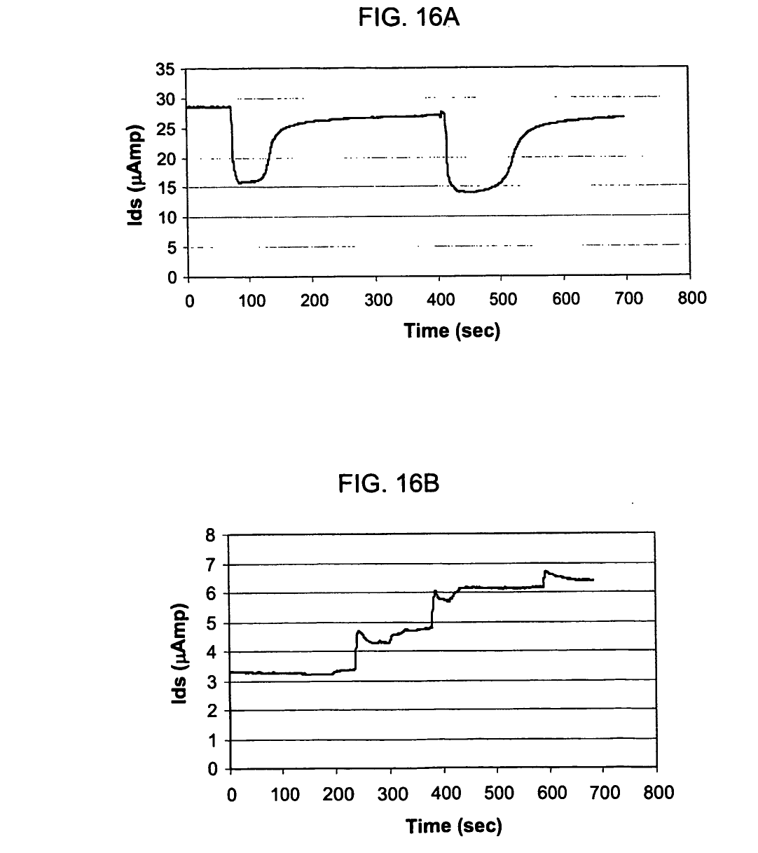

The application of eserine—a reversible acetylcholine esterase inhibitor (Used for medical purposes)—to the solution (without washing ACh) resulted in a major decrease of drain-source current (FIG. 16A). Return of I_{ds} to levels was slightly lower then those before the application of eserine. These results may indicate that the acidification of the solution by the hydrolysis of acetylcholine is halted by eserine only locally, e.g. the esterase activity is halted until sufficient amounts of substrate is diffused to the gate area. Eserine is charged at physiological pH, a fact that could have explained the decrease in I_{ds}. However, the application of eserine onto a non-enzyme-functionalized ISFET has been shown to have opposite effects (FIG. 16B). A minor increase in I_{ds} was recorded which strengthens the suggestion that the effect of eserine is due the inhibition of enzyme activity. Furthermore, the reversible nature of ACHE inhibition by eserine correlates with the current increase and may explain this phenomenon.

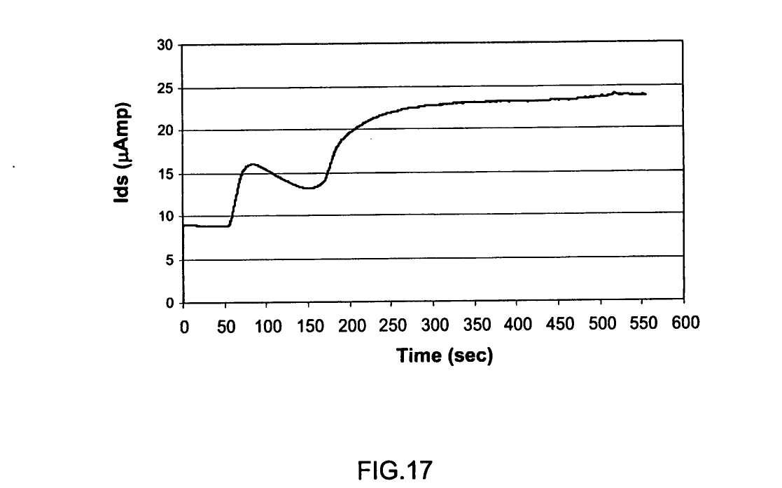

The long-term fidelity of the enzyme-functionalized ISFETs was also analyzed. ISFETs responsiveness has shown no major decrease in after 30 days in 4° C. (FIG. 17). Such high fidelity is non-existent in traditional enzyme based sensors and offers stability and reuse of sensor. The response time of the sensor is reflected by the amount of time it takes for I_{ds} to reach steady state after the application of the substrate. The average response time of the enzyme modified ISFET was measured under different concentration and was found to be approximately 35 seconds. The response time under low substrate concentrations (10 μM-50 μM) was lower then response time under high concentrations (100 μM and above).

1. A device for the detection of analyte molecules, the device comprising at least one pair of source-drain electrodes and at least one gate electrode to thereby define at least one Field Effect Transistor (FET), wherein said at least one gate electrode is coated with a layer of receptor molecules that in the presence of said analytes catalyze a reaction that causes release of ions in a medium surrounding said receptor molecules, and a monolayer of linker molecules is provided for linking said receptor molecules to said at least one gate such that a distance between the receptor molecules layer and the surface of the coated gate is smaller than 15 Å.

2. A device according to claim 1, wherein said distance is of a few angstroms.

3. A device according to claim 1, wherein the Field Effect Transistor is an Ion Sensitive Field Effect Transistor.

4. A device according to claim 1, wherein the receptor molecules are enzymes or peptides.

5. A device according to claim 4, wherein the receptor molecules is acetylcholine esterase.

6. A device according to claim 1, wherein said analyte molecules are selected from chemical agents used in agriculture, in environmental applications, industry and chemical warfare.

7. A device according to claim 6, wherein said chemical agents are pesticides, herbicides, nerve agents and synthetic or natural toxins emitted from industrial plants.

8. A device according to claim 1, wherein said gate electrode is an ion sensitive oxide gate.

9. A device according to claim 8, wherein the ion-sensitive oxide is Aluminum Oxide (Al₂O₃), Silicon Nitride (Si₃N₄), Indium Tin Oxide (In₂O₃—Sn₂O₃), Silicon Oxide (SiO₂) or Tantalum Oxide (Ta₂O₅).

10. A device according to claim 1, wherein said linker molecules are covalently bound to at least one of the surface or the receptor molecules.

11. A device according to claim 8, wherein said linker molecules are selected from conjugated or unconjugated aliphatic, aromatic or heteroaromatic molecules, having at least one functional group capable of covalently binding to said surface and at least one functional group capable of covalently binding to said receptor molecules.

12. A device according to claim 1, comprising an array of gate electrodes each gate electrode being coated with receptor molecules layer different from that of the other gate electrodes.

13. A device according to claim 12, wherein said array of gate electrodes is associated with the same source-drain pair.

14. A device according to claim 12, wherein each of the gate electrodes is associated with a different source-drain pair.

15. A method of detecting analyte molecules in a medium, the method comprising:
(a) providing at least one Field Effect Transistor (FET) formed by a source-drain electrode pair and at least one gate electrode that is coated with a layer of receptor molecules that in the presence of certain analytes catalyze a reaction that causes release of ions in a medium surrounding said receptor molecules, and a monolayer of linker molecules for linking said receptor molecules to said at least one gate such that a distance between the receptor molecules layer and the surface of the coated gate is smaller than 15 Å.
(b) accommodating said at least one FET such that said at least one gate is exposed to a medium suspected of containing analyte molecules capable of reacting with the receptor molecules, thereby affecting a release of ions in said medium, and
(c) monitoring a change in an electric current between the source and drain electrodes caused by the release of ions, said change being indicative of the presence of said analyte in the medium, thereby enabling measuring the analyte concentration in the medium.

16. A method according to claim 15, wherein said medium is one of the following: water, sea water, buffer, and ionic solution.
